(19) United States
(12) Patent Application Publication
(10) Pub. No.: US 2024/0245812 A1
(43) Pub. Date: Jul. 25, 2024

(54) DEVICES AND METHODS FOR MEDICAL DEVICE DISINFECTION

(71) Applicant: Mick Abae, Davie, FL (US)

(72) Inventor: Mick Abae, Davie, FL (US)

(21) Appl. No.: 18/242,182

(22) Filed: Sep. 5, 2023

Related U.S. Application Data
(60) Provisional application No. 63/405,124, filed on Sep. 9, 2022.
(60) Provisional application No. 63/421,420, filed on Nov. 1, 2022.

(51) Int. Cl. A61L 2/10

(52) U.S. Cl. CPC A61L 2/10; A61L 2202/11; A61L 2202/121; A61L 2202/14; A61L 2202/24

(57) ABSTRACT

Devices and methods for disinfecting a medical probe are provided. In an aspect, a disinfection device for a medical probe includes a container having an opening, a plurality of ultraviolet (UV) light emitters disposed in the container and to emit UV light to disinfect a surface of the medical probe, and a probe holder to hold the medical probe apart from the plurality of electromagnetic wave emitters when at least a portion the medical probe is inserted into the opening of the container and the probe holder.

### TECHNICAL FIELD

The present application relates to devices and methods for disinfecting a medical device.

### BACKGROUND

Disinfecting medical devices for repeated use is an issue for patient safety

Use of medical devices can potentially transmit pathogens. Different types of medical devices may require different cleaning methods. Medical devices that are most vulnerable to pathogen transmission may be those intended to penetrate skin or mucous membranes, such as scissors, forceps, tweezers, or hemostats, which may require sterilization. Medical devices that may come into contact with mucous membranes or inner surface of a body, such as intracavitary endocavitary probes such as transducers, may require high levels of disinfection rather than sterilization. Medical devices that come into contact with intact skin but not mucous membranes, such as blood pressure cuffs, a stethoscope, or external transducers, may not require high level disinfection.

Probes for medical uses that directly contact a patient's interior body surfaces such as a mucous membrane may require a high-level of disinfection and may require the application of a high-quality single-use cover during each use, compared to other probes that only contact external surfaces of the body. Proper high-level disinfection may be mandatory to protect patients from potential infection.

### SUMMARY

This Summary is provided to introduce a selection of concepts in simplified form that are further described below in the Detailed Description. This Summary is not intended to identify key aspects or essential aspects of the claimed subject matter.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

As described herein, an aspect of the present disclosure relates to a disinfection device for a medical probe or a method of disinfecting a medical probe.

In various embodiments, the device may comprise a container having an opening, a plurality of ultraviolet (UV) light emitters to emit UV light in the container to disinfect a surface of the medical probe, and a probe holder to hold the medical probe to be apart from the plurality of UV light emitters to provide a distance to disperse the UV light over the surface in the container when at least a portion the medical probe is inserted into the opening of the container and the probe holder.

In various embodiments, the probe holder may hold the medical probe to be substantially perpendicular to a direction of the UV light to be emitted from one of the plurality of UV light emitters.

In various embodiments, the device may comprise a meter to estimate a degree of an exposure of the surface to the UV light. In various embodiments, the meter may include a sensor, a dosimeter, a digital dosimeter, or a timer.

In various embodiments, the UV light may include UVA, UVB, UVC, or a combination thereof.

In various embodiments, a wavelength of the UV light may be from about 10 nm to about 400 nm.

In various embodiments, the device may further comprise a cap to close the opening to block the UV light and a cable accommodating portion to accommodate a cable of the medical probe around the opening, to block the UV light around the cable, when at least a portion of the medical probe is inserted into the opening and the cap closes the opening.

In various embodiments, the device may comprise a vent to vent air inside the container.

In various embodiments, the device may comprise a filter to filter the air vented through the vent.

In various embodiments, the device may comprise a bracket to couple to a portion of a mobile caster holding pole.

In various embodiments, the device may comprise a sensor to obtain information from the medical probe.

In various embodiments, the sensor is to obtain the information from a label or a medium containing the information, such as a memory storing information, a bar code, a QR code, or a RFID chip.

In various embodiments, a method of disinfecting an medical probe may comprise inserting at least a portion of the medical probe, through an opening of a container, into the container containing a plurality of ultraviolet (UV) light emitters to emit UV light to disinfect a surface of the medical probe, inserting at least a portion of the medical probe into a probe holder to hold the medical probe to be apart from the plurality of UV light emitters to provide a distance to disperse the UV light over the surface in the container, and supplying power to the plurality of UV light emitters to emit the UV light to disinfect the surface of the medical probe

In various embodiments, the method may comprise estimating a degree of an exposure of the surface to the UV light using a meter to estimate the degree of the exposure. In various embodiments, the meter may include a sensor, a dosimeter, a digital dosimeter, or timer.

In various embodiments, the UV light includes UVA, UVB, UVC, or a combination thereof.

In various embodiments, a wavelength of the UV light may be from about 10 nm to about 400 nm

In various embodiments, the method may comprise venting air inside the container through a vent.

In various embodiments, the method may comprise filtering the air vented through the vent using a filter.

These and other embodiments will become readily apparent to those skilled in the art from the following detailed description of certain embodiments having reference to the attached figures; the invention not being limited to any particular embodiment disclosed.

### BRIEF DESCRIPTION OF THE FIGURES

FIG. 1 illustrates an embodiment of a device to disinfect a medical probe; and

FIG. 2 illustrates an embodiment of a device having a holder to hold a plurality of medical probes to disinfect the plurality of medical probes.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting embodiments and the invention may be practiced according to the claims without some or all of these specific details. For the purpose of clarity, technical material that is known in the technical fields related to the invention has not been described in detail so that the invention is not unnecessarily obscured.

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

The term “patient”, as used herein, refers to an individual with his/her portion of body such as a body surface to be contacted by a medical device such as a medical probe.

Other embodiments of implementations will become apparent to the person skilled in the art in view of the teachings of the present description and as such, will not be further described here.

Note that tides or subtitles may be used throughout the present disclosure for the convenience of the reader, but in no way should these limit the scope of the invention. Moreover, certain theories may be proposed and disclosed herein; however, in no way should they, whether they are right or wrong, limit the scope of the invention so long as the invention is practiced according to the present disclosure without regard for any particular theory or scheme of action.

Any and all references cited throughout the specification are hereby incorporated by reference in their entirety for all purposes.

It will be understood by those of skill in the art that throughout the present specification, the term “a” used before a term encompasses embodiments containing one or more of what the term refers to. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions, will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

In various embodiments, a medical device such as a medical probe including a probe such as a transducer for an intracavitary contact or an endocavitary contact may be cleaned using high level disinfection and be used in conjunction with a single-use transducer cover. For example, a disinfection, a cover, or a combination thereof must warrant protection against human viruses, including human immunodeficiency virus, human papillomavirus (HPV), hepatitis B, and others.

Disinfection of a medical device including an intracavitary probe and an endocavitary probe, such transvaginal, transrectal, intraoperative and transesophageal probes, requires high level disinfection before they can be used on another patient.

For disinfecting, a chemical agent can be used. However, chemical based disinfection may not be fully effective in disinfecting a medical probe in some cases. For example, there is a possibility of a human error not properly following a given protocol. For example, depending on the employed disinfection agent, a medical probe may need to be thoroughly rinsed and dried after disinfection, following manufacturer guidelines. High-level chemical disinfectants may require clean and dry surfaces as wet surfaces dilute the disinfectant. For a chemical disinfectant or agent, precautions must be taken to protect workers and patients from the toxicity of the disinfectant. High-level liquid disinfection may be required to ensure further statistical reduction in the microbial load. Depending on a chemical type, immersion of a medical probe in fluids may be required, which demands medical probe's ability to be submerged. After disinfection process, a stored medical probe may be recontaminated before use.

A medical probe such as an intracavitary probe or endocavitary probe transducers have been found to be persistently contaminated despite the high-level disinfection procedure or use of a probe cover and that that a medical probe may carry pathogens, including HPV, unless properly disinfected between examination sessions.

In various embodiments, a device for disinfecting a medical probe is provided. A device for disinfecting a medical probe may provide additional protection for patient safety. A device for disinfecting a medical probe may allow a storage of a medical probe in a disinfecting stage until the device is used, thereby reducing the risk of recontamination.

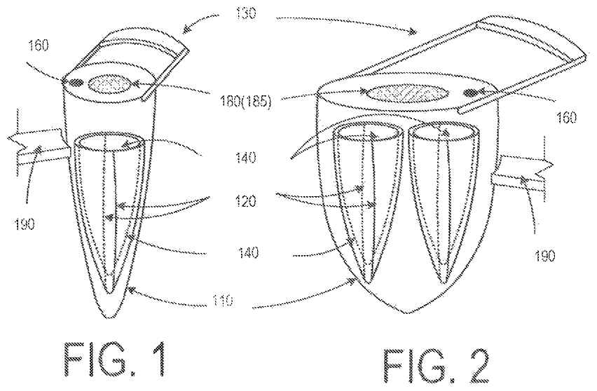

For example, FIG. 1 illustrates a device 100 for disinfecting a medical probe. Referring to FIG. 1, the device may include a plurality of ultraviolet (UV) light emitters 120 to emit UV light in the container 110 to disinfect a surface of the medical probe.

In various embodiments, the container 110 may include a rigid portion and/or a flexible portion. For example, the container 110 may be a rigid containing portion or a pouch with a flexible material. For example, a rigid portion may be used to provide a machinal protection to a medical probe while a flexible portion may provide an additional accommodation for placing the device 100 housing a medical probe. For example, the container 110 may include a portion constructed with a flexible and durable ultraviolet resistant material such as a polycarbonate silicone rubber material. In various embodiments, the container 110 may include an opening through which at least a portion of a medical probe can be inserted for disinfection by the device 100. In various embodiments, the container 110 may be in a variety of shapes such as a cylindrical shape or a bar shape to accommodate the shape of a medical probe. In various embodiments, the container may be in a shape to facilitate an insertion of a medical probe. For example, the cross-sectional area around the opening may be larger than another cross-sectional area of the container further away from the opening along the length direction of the container 110. For example, the container may be substantially in a cone shape, a funnel shape or a pyramidal shape, to provide a wider opening area.

In various embodiments, the device 100 may comprise a cap 130 or an operating cap 130 to close the opening of the container 110 to block the UV light and a cable accommodating portion to accommodate a cable of the medical probe around the opening, to block the UV light around the cable, when at least a portion of a medical probe is inserted into the opening and the cap closes the opening.

In various embodiments, the device 100 may include an operating cap 130 to open and close the opening of the container 110. In various embodiments, the operating cap 130 may be design to fully close the opening without any gap to block the UV light being emitted. For example, the operating cap 130 and/or the opening may have a seal or lining to suppress or avoid a gap formation in between the cap 130 and the opening. In various embodiments, the operating cap 130 and/or the opening may include an accommodating portion to accommodate a cable attached to a medical probe to suppress or avoid a gap formation in between the cap 130 and the opening. For example, the operating cap 130 and/or the opening may include a recess or an indent to which at least a part of the cable can be inserted or accommodated. For example, the operating cap 130 and/or the opening may include a soft or flexible rubber seal that can be deformed around the cable to suppress or avoid a gap formation in between the cap 130 and the opening. In various embodiments, the operating cap 130 may close the opening in a locked position to avoid UV light leakage. According to closing and opening of the operating cap 130 may activate or deactivate the UV light emission from the plurality of the UV light emitters 120, for example, to provide a safety measure to the UV light. In various embodiments, closing a cap may activate the UV light emission for a period of the time, for example, using a timer to control power supply to the plurality of UV light emitters 120. This way, the closing state with the UV light off may notify that the medical probe contained has been disinfected by a set period of the time and has been sealed from recontamination within the container because the cap 130 was not opened after the disinfection for the set period. Such a closed state after the period of the disinfection may be indicated by a medium such as an indication light or sign.

In various embodiments, a UV light emitter 120 may include a UV light source. A UV light source may include a variety of light sourcetypes, such as a lightbulb, fluorescent light, LED light such a LED light strips. In various embodiments, A UV light source may be coated with additional fluorescent materials, for example, to add or adjust a wavelength of the UV light emitted by the emitter. In various embodiments, a UV light disperser or lens may be provided, for example, to focus or disperse the UV light. For example, a UV light disperser or lens may focus or disperse the UV light based on the distance between the UV light emitter and a surface of a medical probe to be disinfected by the UV light.

In various embodiments, the device 100 may be internally lined by four equidistantly separated installed UV-C Flex-LED strip lights to produce 360 degrees of UV-C light exposure for disinfecting. For example, the disinfecting zone of the device will be created with the LED strip lights length of approximately 42.5 cm and maximum width of approximately 15 cm within the interior compartment of the device.

In various embodiments, the device 100 may include a probe holder 140 to hold the medical probe, for example to keep the medical probe to be apart from the plurality of UV light emitters 120, to provide a sufficient distance to disperse the UV light over the surface of the medical probe in the container 110 when at least a portion the medical probe is inserted into the opening of the container 110 and the probe holder 140.

In various embodiments, the device 100 may comprise a meter 160 to estimate a degree of an exposure of the surface to the UV light. In various embodiments, the meter 160 may include a sensor, a dosimeter, a digital dosimeter, or a timer. In various embodiments, a different location may be selected as a location of the meter 160. For example, the location of the meter 160 may be mobile and may be on a surface of a medical probe to measure the degree of exposure of the surface to the UV light. The meter 160 may be placed at or around a location with relatively lower UV light exposure such as a shaded area or an area not directly facing a UV light emitter 120. For example, the location of the meter 160 may be at or around the bottom or the bottom tip of the container 110 or on an internal side of the operating cap 130. For example, an UVC dosimeter card may be used to provide visible evidence of successful disinfection.

In various embodiments, the device 100 may include a reader to read or detect an indicator from the medical probe, which may be, for example, a sensor or a communicator device. For example, the medical device or the medical probe may include an indicator in a variety of forms, which is to contain information related to the status of the medical device or the medical probe. A variety of forms of the indicator may include form of a label, such as a bar code or a QR code. A variety of forms of the indicator may include form of information storage, such as a machine-readable medium, chip or memory that stores the information, such as radio-frequency identification RFID-based indicator, or other form of communication device that may include a processor, memory and a communication module. The device 100 may include a reader in a variety of forms to read information from the indicator, such as a sensor or a camera to read the indicator, or a communication device to read information from the information storage.

For example, the device 100 may include a bar code scanner or reader added for the purpose of identification of the unique bar code identifier assigned for each medical device or each medical probe such as endocavitary ultrasound probe. Each medical device such as a medical probe to be disinfected will be provided with a specific bar code label such as a bar code unique to the medical device, which will be secured to the medical device. The bar code scanner will then record and register, e.g., in a log format, the device's unique identifier that is to be disinfected, being disinfected, or having been disinfected, the time a date of the disinfection, and/or the satisfactory level of disinfection obtained by the meter 160, such as a timer or a dosimeter. This electronic logging feature can automate and replace the manual logging processes that are required for documentation purposes for each time the specific medical device that is used and has undergone the disinfection process.

In various embodiments, the U V light may include UVA, UVB, UVC, or a combination thereof. In various embodiments, a wavelength of the JV light may be from about 10 nm to about 400 nm.

In various embodiments, the device 100 may comprise a vent 180 to vent air inside the container. The device 100 may comprise a fan to evacuate air inside the container 110. The device 100 may comprise a filter 185 to filter the air vented through the vent 180. For example, the filter 185 may include activated carbon filter, to filter our odor and/or a by-product generated from the UV light such as a radical or ozone.

In various embodiments, the device 100 may comprise a bracket 190 or a mounting bracket 190 to couple to a portion of a mobile caster holding pole.

In various embodiments, the device may include a plurality of medical probe holders to disinfect a plurality of medical probes. For example, the plurality of medical probe holders may be provided within the container 110 along with respectively corresponding UV light emitters. For example, FIG. 2 illustrates a device 100 having two medical probe holders to hold two medical probes for disinfection.

In various embodiments, a method of disinfecting a medical probe may comprise inserting at least a portion of the medical probe, through an opening of a container 110, into the container 110 containing a plurality of ultraviolet (UV) light emitters 120 to emit UV light to disinfect a surface of the medical probe. The method may comprise inserting at least a portion of the medical probe into a probe holder 140 to hold the medical probe to be apart from the plurality of UV light emitters 120 to provide a distance to disperse the UV light over the surface in the container 110, and supplying power to the plurality of UV light emitters 120 to emit the UV light to disinfect the surface of the medical probe.

In various embodiments, the method may comprise estimating a degree of an exposure of the surface to the UV light using the meter 160 to estimate the degree of the exposure. In various embodiments, the UV light includes UVA, UVB, UVC, or a combination thereof.

In various embodiments, the method may comprise venting air inside the container 110 through a vent 180.

In various embodiments, the method may comprise filtering the air vented through the vent 180 using a filter 185.

What is claimed is:

1. A disinfection device for a medical probe, comprising:
a container having an opening;
a plurality of ultraviolet (UV) light emitters for emitting UV light in the container to disinfect a surface of the medical probe; and
a probe holder to hold the medical probe apart from the plurality of UV light emitters to provide a distance to disperse the UV light over the surface in the container when at least a portion the medical probe is inserted into the opening of the container and the probe holder.

2. The device according to claim 1, wherein the probe holder is configured to hold the medical probe to be substantially perpendicular to a direction of the UV light emitted from one of the plurality of UV light emitters.

3. The device according to claim 1, further comprising a meter to determine a degree of exposure of the surface to the U V light.

4. The device according to claim 3, wherein the meter comprises a sensor, a dosimeter, a digital dosimeter, or a timer.

5. The device according to claim 1, wherein the UV light comprises UVA, UVB, UVC, or a combination thereof.

6. The device according to claim 1, wherein a wavelength of the UV light is from about 10 nm to about 400 nm.

7. The device according to claim 1, further comprising:
a cap to close the opening, and
a cable accommodating portion to accommodate a cable of the medical probe around the opening, to block the UV light around the cable, when at least a portion of the medical probe is inserted into the opening and the cap closes the opening.

8. The device according to claim 1, further comprising a vent to vent air from the container.

9. The device according to claim 8, further comprising a filter to filter the air vented through the vent.

10. The device according to claim 1, further comprising a reader coupled to the container to obtain information from the medical probe.

11. The device according to claim 10, wherein the reader is to obtain the information from a label indicating the information, a memory device storing the information, a bar code, a QR code, or a RFID chip.

12. A method of disinfecting a medical probe, comprising:
inserting at least a portion of the medical probe, through an opening of a container, into the container containing a plurality of ultraviolet (UV) light emitters to emit UV light to disinfect a surface of the medical probe;
inserting at least a portion of the medical probe into a probe holder to hold the medical probe to be apart from the plurality of UV light emitters to provide a distance to disperse the UV light over the surface in the container; and
supplying power to the plurality of UV light emitters to emit the UV light to disinfect the surface of the medical probe.
